(19) United States
(12) Patent Application Publication
(10) Pub. No.: US 2008/0147174 A1
(43) Pub. Date: Jun. 19, 2008

(54) Apparatus and method of using markers to position stents in bifurcations

(75) Inventors: Eitan Konstantino, Orinda, CA (US); Tanhum Feld, Moshaw Merhawya (IL)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: TriReme Medical, Inc., Pleasanton, CA (US)

(21) Appl. No.: 12/001,763

(22) Filed: Dec. 11, 2007

Related U.S. Application Data
(60) Provisional application No. 60/869,515, filed on Dec. 11, 2006.

(51) Int. Cl. A61F 2/06

(52) U.S. Cl. 623/1.35

(57) ABSTRACT

The present invention provides methods and systems for placing stent systems at vascular bifurcations. The systems include a main branch stent having a side opening, optionally including a side structure, with radiopaque fluoroscopic markers about the periphery of the opening. The stent system further includes a side branch stent having radiopaque markers near at least end thereof. The main branch stent is positioned in the main blood vessel lumen using the markers for proper positioning. After deploying the main branch stent, the side branch stent is positioned through an opening within the side branch. Using the markers on both the main branch stent and the side branch stent, proper alignment and positioning of the two stents relative to each other may be achieved.

### CROSS REFERENCES TO RELATED APPLICATIONS

The present application claims the benefit of provisional application No. 60/869,515 (Attorney Docket No. 022246-000800US) filed on Dec. 11, 2006, the full disclosure of which is incorporated herein by reference.

### BACKGROUND OF THE INVENTION

### 1. Field of the Invention

The present invention relates in general to medical devices and more specifically to medical devices used in the treatment of vascular stenoses at or near a bifurcation lesion.

Stenting is a common medical procedure intended for revascularization of stenotic vessels where a blocked artery is dilated and a stent is placed in the artery to maintain vessel patency following the procedure. A stent is small mesh like tubular device, usually fabricated from metal, that can optionally be coated with a drug or a polymer containing a drug.

While stents are successful in treating a variety of lesions in the vascular system, their success has been limited in the treatment of bifurcation lesions and ostial lesions. Often, when a stent is deployed in a main vessel at a bifurcation, the stent blocks access to the side branch thereby disrupting blood flow patterns and limiting blood flow to the side branch. To address the problem, stents with a side window, a side opening or a side branch support structure are used. Such asymmetrical stent structures require rotational and axial alignment to register the side opening with the side branch ostium.

Of particular interest to the present invention, an additional stent may be placed at or through the side opening into the side branch, usually when the side branch is diseased. Placement of such “side branch stents” often leaves a gap between the main branch and side branch stents. Restenosis often occurs in this gap. Drug eluting stents often fail to inhibit restenosis at bifurcation lesions. This failing is attributed to the lack of metallic stent coverage in the gap between the main vessel stent and the side branch stent.

The gap may be eliminated by delivering the side branch stent with a portion thereof protruding into the main vessel. The protruding portion of the stent will be crushed by expanding a balloon in the main vessel, often during delivery and expansion of the main vessel stent. While effective in some instances, crushing the side branch stent can lead to undesired deformation of the stent as well as dissection of the blood vessel.

For these reasons, it would be desirable to provide improved methods and systems for vascular stenting at bifurcations. In particular, it would be desirable to provide methods and systems which provide a more complete coverage by a stent structure in the region of the ostium between a main blood vessel and a side branch blood vessel. At least some of these objectives will be met by the inventions described hereinafter.

### SUMMARY OF THE INVENTION

The present invention provides methods and devices for using asymmetrical radiopaque marker configurations for the alignment and positioning of asymmetrical main branch stents and positioning of a side branch stent in a side branch vessel in association with placement of a main branch stent in the main vessel. The term “bifurcation” in this patent includes all types of bifurcation lesions and lesions near bifurcations in the vessels. The phrases “bifurcation ostium area” and “ostial lesion” apply to all types of lesions including those located at aorto-ostial and anastomosis sites.

Methods according to the present invention for deploying a stent structure at a bifurcation between a main vessel and a side branch vessel comprise deploying a first or main branch stent in the main vessel so that a side opening of the stent is positioned at least partially over an ostium of the side branch. The side opening of the stent has at least one fluoroscopic marker adjacent to a periphery of the side opening, typically having at least two fluoroscopic markers, often having four or more fluoroscopic markers surrounding or otherwise attached to the periphery. As the markers are located adjacent to the periphery, they will be asymmetrical with respect to an axis of the stent, allowing a user to fluoroscopically view the marker(s) and rotationally and axially align the stent in the lumen of the main branch vessel with the ostium of the side branch vessel prior to deployment of the first stent.

After deploying the first stent, a second stent is positioned through the side opening of the first stent. The second stent has at least one second fluoroscopic marker located near a proximal end of the stent, typically having at least two fluoroscopic markers and optionally having four or more fluoroscopic markers. The second marker(s) may be formed on or attached to the second stent but will more usually be on a delivery catheter which carries the stent. The fluoroscopic markers on the second stent are aligned with the one or more markers on the first stent prior to deploying the second stent in the side branch vessel. By maintaining the marker alignment, the proximal end of the second stent can be positioned properly relative to the first stent in order to maximize the stent coverage over the region of the side branch vessel proximate the ostium, while minimizing any protrusion of the second stent into the lumen of the main branch vessel.

In a preferred embodiment of the methods of the present invention, the side opening of the first stent will comprise a side structure which opens into the side branch to cover the ostium of the side branch. While in some instances the second or side branch stent may be deployed independently of the first stent itself, for example by using a balloon placed within the side structure, it will be particularly preferred to use self-opening side structures as described, for example, in copending application Ser. Nos. 11/330,382 (Attorney Docket No. 022246-000240US), filed on Jan. 10, 2006; 11/406,139 (Attorney Docket No. 022246-000310US), filed on Apr. 17, 2006; and 11/439,707 (Attorney Docket No. 022246-000410US), filed on May 23, 2006, the full disclosures of which are incorporated herein by reference. In all cases, the at least one fluoroscopic marker of the first stent will be positioned on the side structure so that the marker will usually open through the ostium and into the proximal portion of the side branch lumen after the side structure has been deployed.

In the illustrated embodiments, the first stent is deployed using a balloon catheter for expansion of the stent, where the side structure opens in response to deployment of the first stent. The second stent is then positioned through the side structure using a second balloon catheter. Alternatively, the second stent may be a self-expanding stent which may be positioned by releasing the second stent from constraint at a desired position within the side branch lumen. In either case, before deploying the second stent, the user will axially and rotationally position the second stent within the side branch lumen so that the markers at the proximal end of the second or side branch stent are properly aligned with the previously deployed markers on the first or main branch stent. Usually, the markers will be aligned to overlap each other. Alternatively, the markers may be aligned in any position which has been pre-selected to indicate that the positions of the two stents within the main branch vessel and side branch vessel are proper.

In another aspect of the present invention, a stenting system comprises a main branch stent and a side branch stent. The main branch stent has a side opening for alignment with an ostium of a side branch in a patient's vasculature. One or more first fluoroscopic markers are positioned near a periphery of the side opening to allow for axial and rotational positioning of the main branch stent prior to deployment. The side branch stent has an end which is adapted to be positioned at the side branch opening of the main branch stent. In particular, the end is adapted so that it may be positioned immediately adjacent to the side opening, or more usually within a side structure of the side opening after deployment of the main branch stent. The adapted end of the side branch stent includes one or more second fluoroscopic markers which allow the side branch stent to be aligned with the marker(s) on the main branch stent in order to properly position the two stents relative to each other at a vascular bifurcation. The second markers may be on the second stent but will more usually be on a delivery catheter which carries the second stent.

In the exemplary systems herein, the main branch stent includes a side structure adapted to open laterally from the main branch stent, typically being formed as part of the body of the main branch stent so that it lies flat within or over the body prior to deployment. In those cases, the at least one fluoroscopic marker will be positioned on the side structure so that it will open into the side branch lumen when the side structure is deployed.

In a preferred aspect of the stenting system of the present invention, the side structure opens at least partially in response to radial expansion of the body of the main branch stent. Alternatively, however, the side structure of the main branch stent could open in response to a separate action, such as balloon expansion within the side opening to deploy the structure. In the self-deploying embodiments, the side structure typically comprises at least one wing that opens laterally, more typically comprising two or more wings which are interlinked to open together in response to expansion of the main branch stent. For example, the side structure may include a leverage mechanism that transfers displacement and expansion forces from the main body to open the side structure during radial expansion of the main body stent.

The fluoroscopic markers on the delivery catheter and/or side branch stent are typically positioned at one end of the side branch stent, usually being positioned to align with the at least one marker on the side structure after the side structure is opened.

### BRIEF DESCRIPTION OF THE DRAWINGS

FIGS. 1A and 1B illustrate a main vessel stent having a self-deploying side structure which is useful in the methods and systems of the present invention. FIG. 1A shows the stent with the side structure on the face of the figure. FIG. 1B illustrates the side structure on the top of the figure, with the deployed side structure being shown in broken line.

FIG. 2 is a schematic illustration of a side branch stent useful in the methods and systems of the present invention, shown in its constrained (pre-deployed) configuration in full line and in its expanded (deployed) configuration in broken line.

FIGS. 3A through 3F illustrate deployment of the stents of FIGS. 1A, 1B, and 2 at a bifurcation in the vasculature of a patient using first and second balloon deployment catheters.

FIG. 4 shows a view of the main branch stent system shown in FIGS. 1A and 1B under fluoroscopy at incorrect rotational alignment before expansion where the four markers are visible.

FIG. 5 shows a view of the main branch stent shown in FIGS. 1A and 1B under fluoroscopy at correct rotational alignment before expansion where only two markers are visible.

FIG. 6 shows a view of the main branch stent shown in FIGS. 1A and 1B under fluoroscopy after expansion.

FIGS. 7A and 7B show a view of the bifurcation stent shown in FIGS. 1A and 1B under fluoroscopy after a side branch stent was placed in the side branch.

### DETAILED DESCRIPTION OF THE INVENTION

The current clinical practice of stenting utilizes angiographic images to navigate and deploy stents. Those images are two dimensional and are generated using x-ray radiation. The image can be taken from multiple angles and positions. Radiopaque or fluoroscopic markers have been used to mark ends and other parts of stent delivery systems, angioplasty balloons and guidewires. By using asymmetrical markers and/or asymmetrical configuration of symmetrical markers (i.e. spherical), the present invention enables the alignment of asymmetrical stents using angiography. One example is a stent with a side opening where the side opening in intended to be aligned with the bifurcation or side branch ostium. One or more radiopaque fluoroscopic markers can be attached around the periphery of the side opening or to the struts covering or protruding into the side branch ostium area. Two radio-opaque markers can be attached side by side close the side opening of the stent (rather then the distal or proximal ends of the opening). This will allow the physician to see not only where the side opening is positioned axially, but also the rotational position of the side opening versus the bifurcation ostium. After the first stent is placed, a second stent may be inserted into the side branch, and these markers will help position the second stent. In accordance with the present invention, the second or side branch often will have a marker near a proximal end adapted to or deployed at or in the side opening of the first or main branch stent.

Alternatively a single marker with no symmetry or symmetry in at least one preferred axis and not more than two axes can be used for the same purpose. Projection of this marker to two dimensional image will result in a different shape depending from which side of the marker the x-ray detector is placed. If the position of the x-ray detector is temporarily fixed, the shape projected will give the operator information on the relative position of the marker to the anatomy, typically as the operator rotates the stent, a different image of the marker will show on the screen. For example, a round marker will show rectangular shape when seen from the side.

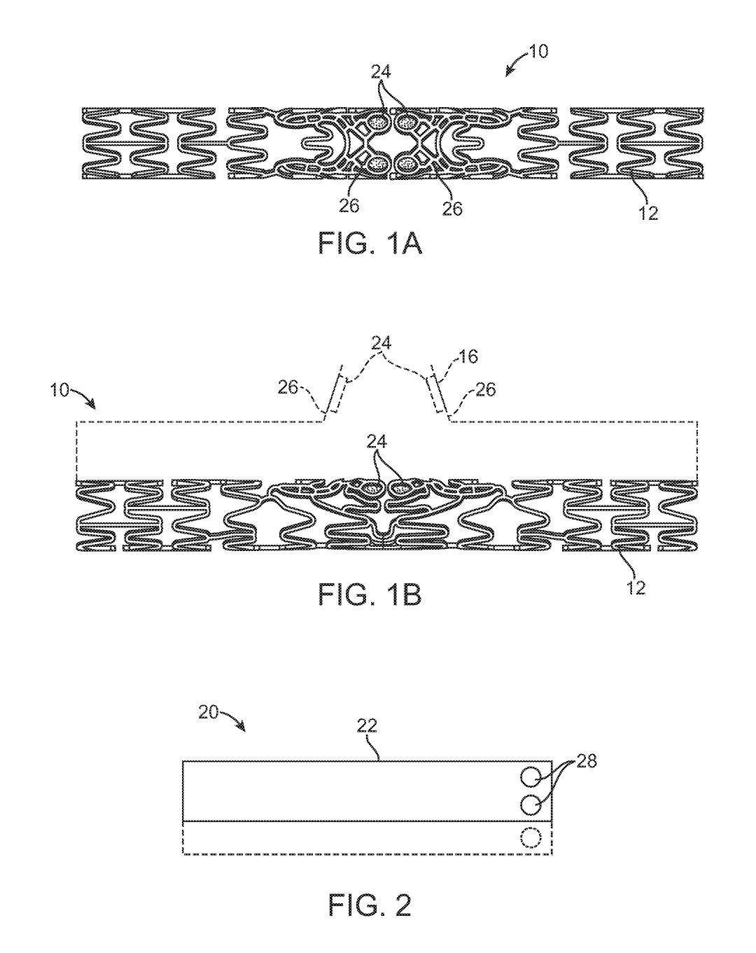

Referring now to FIGS. 1A and 1B, a first or main branch stent 10 suitable for use in the systems and methods of the present invention comprises a tubular main stent body 12 including serpentine rings joined by axial struts in a generally conventional manner. It will be appreciated that the main body 12 of the stent 10 could comprise any one of a number of conventional or newly-developed scaffold structures which may be expanded from an initial reduced diameter configuration, as shown in full line in both FIGS. 1A and 1B, to an expanded deployed configuration as shown in broken line in FIG. 1B.

Of particular interest to the present invention, the stent 10 will include a side opening 16, shown in broken line in FIG. 1B. While the side opening may be of any conventional type used previously in the art, it will preferably be a side structure which opens laterally into a side branch vessel, either as or subsequent to radial expansion of the main body 12. Stent 10 illustrated in FIGS. 1A and 1B has a self-opening side structure of the type generally described in copending U.S. patent application Ser. No. 11/330,382, the full disclosure of which has previously been incorporated herein by reference.

The stent systems of the present invention will also comprise a second or side branch stent 20, as schematically illustrated in FIG. 2. The side branch stent 20 will also include a tubular body 22 which is adapted to be expanded from a radially reduced profile, as shown in full line, to a radially expanded profile, as shown in broken line. Usually, the stent 20 will be balloon-expandable, but in other instances may be self-expanding, typically composed of an elastic material, such as nickel-titanium alloy or other shape memory materials.

Of particular interest to the present invention, the main branch stent will include one or more first radiopaque fluoroscopic markers 24 which surround the side opening 16 after deployment. As shown in FIGS. 1A and 1B, the fluoroscopic or radiopaque markers 24 are disks placed in openable wings 26 which define the side opening. It will be appreciated that the radiopaque fluoroscopic markers 24 are asymmetrically disposed on the stent body 12 so that four markers will appear when the body is in a first rotational configuration, as shown in FIG. 1A, while only two asymmetrically positioned markers will appear when the stent body is rotated 90°, as shown in FIG. 1B. Similarly, the second or side branch stent 20 will have at least one second radiopaque fluoroscopic marker positioned at one end thereof, typically having at least two markers 28, and often having four or more markers. Additional markers may be placed elsewhere along the stent body, but such additional markers are not necessary for the systems and methods of the present invention. Usually the markers will be formed on the balloon or other delivery catheter which carries the second stent.

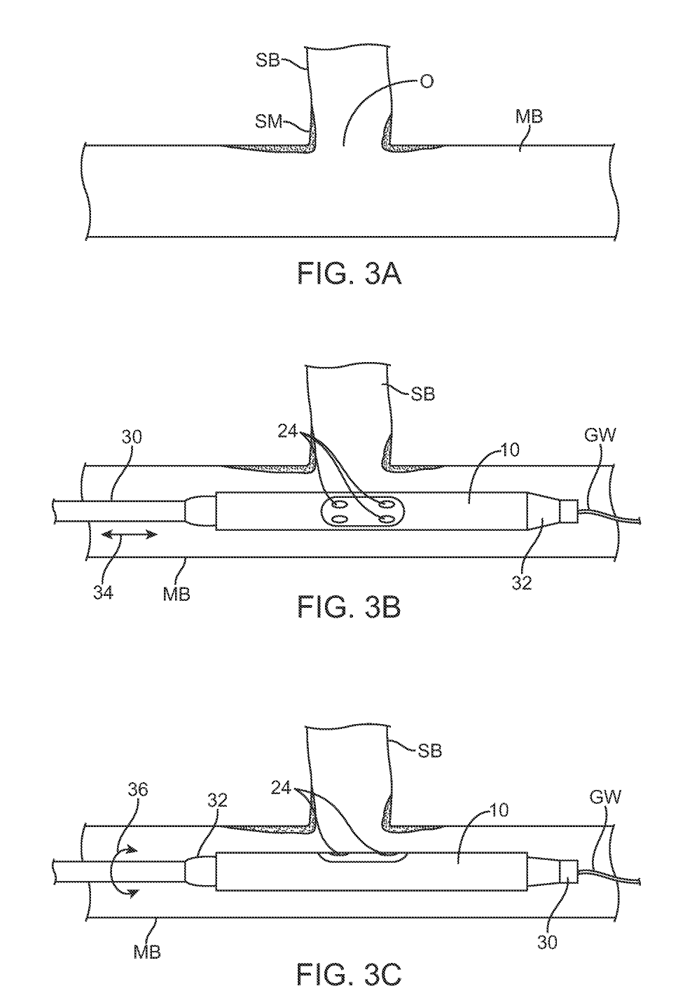

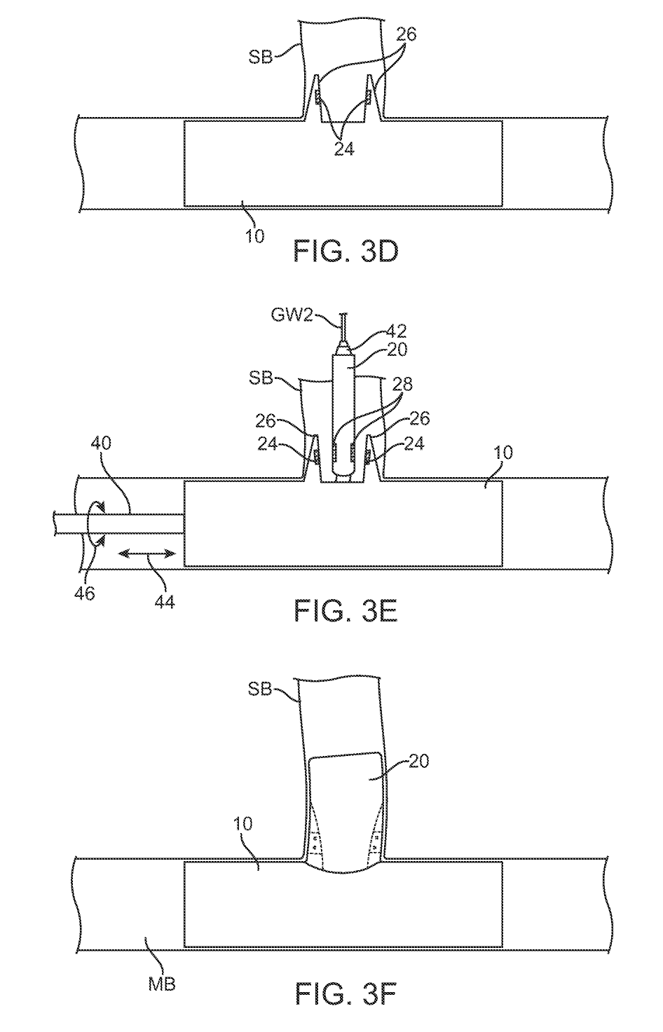

Referring now to FIGS. 3A-3F, deployment of a stent system including both a first or main branch stent 10 and a second or side branch stent 20 at a vascular bifurcation, as shown in FIG. 3A, will be described. The vascular bifurcation includes both a main branch vessel MB and a side branch vessel SB. Stenotic material SM will typically be found at an ostium O which comprises the opening in a wall of the main branch vessel to the side branch vessel.

The first or main branch stent 10 may be delivered to the region of the ostium O when the delivery catheter 30 where the stent is placed on an expandable balloon 32. Delivery will typically be over a guidewire GW. As shown in FIG. 3, the stent 10 is not properly aligned rotationally. After rotating the stent 90°, as shown in FIG. 3C, the stent 10 may be expanded in order to deploy the wings 26 of the side branch structure into the side branch vessel SV, as shown in FIG. 3D. It will be appreciated that as the catheter 30 is introduced, it may be both advanced and retracted axially in the direction of arrow 34 in FIG. 3B, as well as rotationally, as shown by arrow 36 in FIG. 3C. After the first or main branch stent 10 has been deployed, as shown in FIG. 3D, a second or side branch stent 20 may be advanced through the side opening between wings 26, as shown in FIG. 3E. Stent 20 may be delivered by catheter 40 on balloon 42 over guidewire GW. Before expanding the second stent 20, the catheter 40 will be advanced in the direction of arrow 44 and rotated in the direction of arrow 46 in order to position the fluoroscopic radiopaque markers 28 so that they align with the markers 24 on the first stent 10. Once the markers 26 and 28 are aligned (which is illustrated as being immediately opposed and adjacent to each other but could be in other configurations), the stent 20 may be expanded by inflating balloon 42, leaving the final stent structure including both stent 10 and stent 20, as illustrated in FIG. 3F. By properly aligning the first stent 10 and second stent 20 prior to expansion of the second stent, the gap between the first and second stents may be minimized.

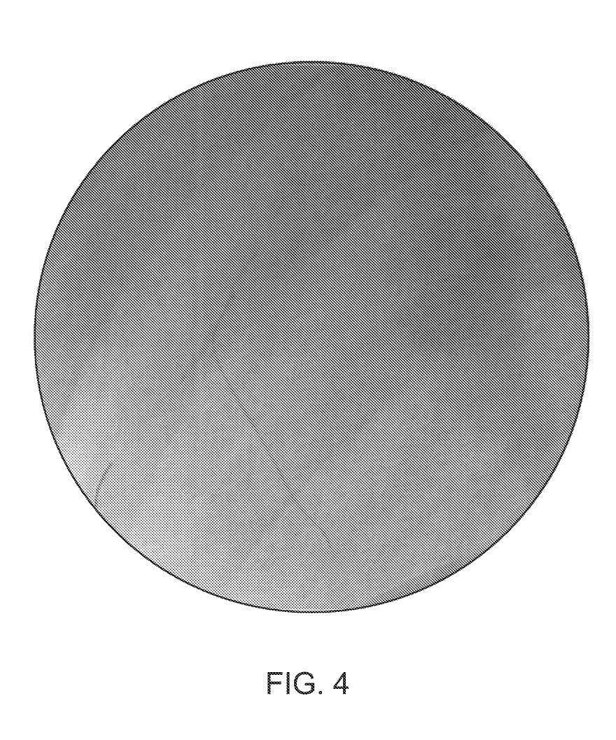

An exemplary view of the stenting system of the present invention via fluoroscopy is shown in FIG. 4 where the stent of FIGS. 1A and 1B mounted on a delivery catheter is viewed when delivered to an arterial bifurcation. Four visible radiopaque markers as shown in FIG. 4 indicate that the stent is not rotationally aligned with the ostium of the side branch. While the system in FIG. 4 shows two guidewires, it is beneficial to use one guide wire system and align the stent using the markers alone. The profile of the system can be significantly reduces to diameter of less than 0.040 inches, often less than 0.030 inches, if a fixed guidewire is used on the catheter delivering the main branch stent.

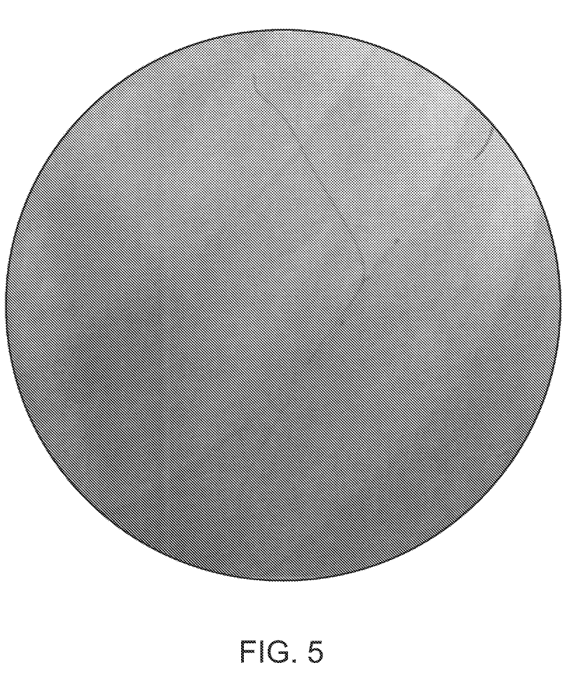

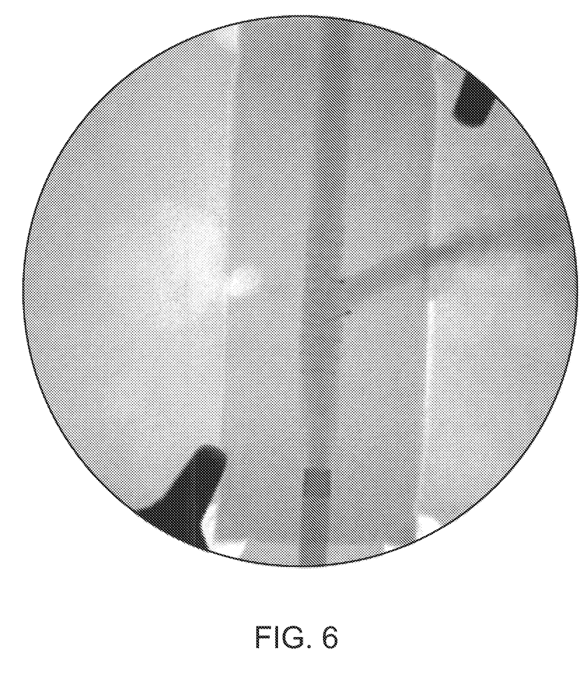

When the stent is rotated to the desired position, only two radiopaque markers are visible, indicating that the stent is rotationally aligned with the ostium of the side branch as shown in FIG. 5.

FIG. 6 shows the stent of FIGS. 4 and 5 after expansion in an arterial bifurcation with two visible radiopaque markers that are aligned with the ostium of the side branch. In case there is a need to insert a guide wire into the side branch the physician can insert the guide wire into the side branch in between the radiopaque markers

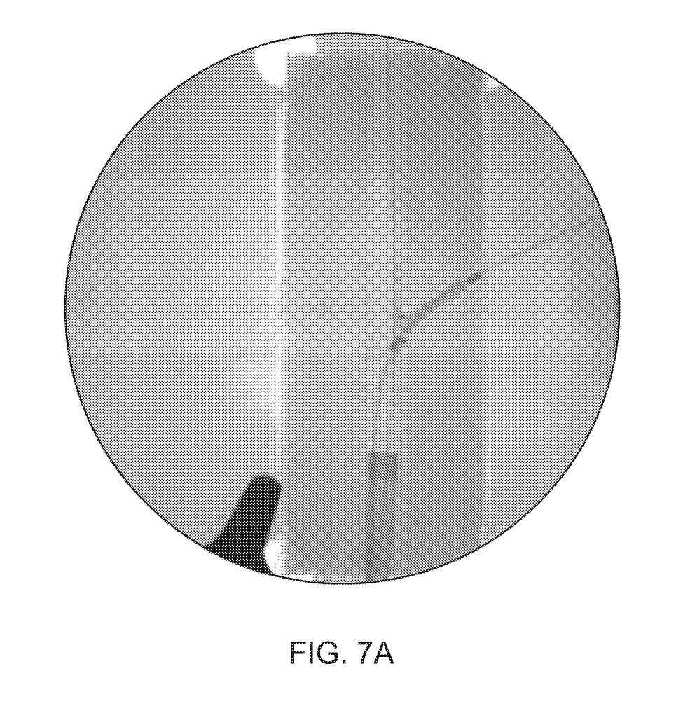

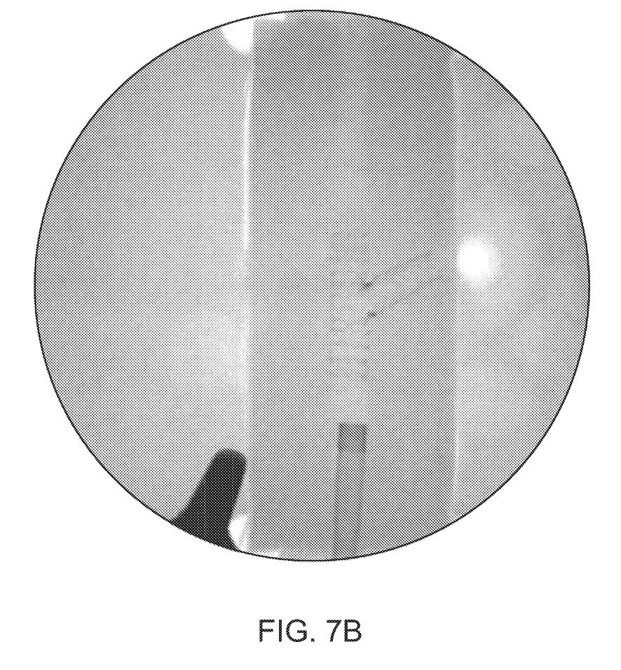

Placement of a side branch stent in the side branch is shown in FIGS. 7A-7B. In FIG. 7A the markers of the side branch stent delivery system are placed next to the markers of the main vessel stent (previously shown in FIG. 6). FIG. 7B shows the result after deployment of the side branch stent. Using the markers, it is possible to avoid gaps or protrusion into the main vessel by the side branch stent.

The second stent catheter normally has two radiopaque markers attached to the catheter at both ends of the stent. Aligning the second stent proximal marker with the side portion markers will result in accurate placement as shown in FIGS. 7A and 7B. If the side portion does not include radiopaque markers the second stent may be placed either too distally creating a gap between the bifurcation stent and the second stent or too proximally protruding into the main vessel. Both are potential causes for clinical problems such as restenosis and thrombosis.

While the above is a complete description of the preferred embodiments of the invention, various alternatives, modifications, and equivalents may be used. Therefore, the above description should not be taken as limiting the scope of the invention which is defined by the appended claims.

What is claimed is:

1. A method for deploying a stent structure at a bifurcation between a main vessel and a side branch vessel, said method comprising:
deploying a first stent in the main vessel so that a side opening of the stent is positioned at least partially over an ostium of the side branch, wherein the side opening of the stent has at least one fluoroscopic marker adjacent to a periphery thereof;
positioning a second stent through the side opening of the first stent, wherein at least one second fluoroscopic marker is located near a proximal end thereof, such that the at least one fluoroscopic marker of the second stent is aligned with the at least one marker of the first vessel stent; and
deploying the second stent in the side branch vessel while maintaining the marker alignment.

2. A method as in claim 1, further comprising opening a side structure from the side opening into the side branch to cover the ostium.

3. A method as in claim 2, wherein the at least one fluoroscopic marker of the first stent is on the side structure and positioned near the ostium in the side branch after the side branch is opened.

4. A method as in claim 3, wherein the second stent is positioned after the side structure has been opened.

5. A method as in claim 2, wherein the side structure opens in response to deployment of the first stent with a first balloon.

6. A method as in claim 5, wherein the second stent is deployed with a second balloon introduced after the first balloon has been removed.

7. A method as in claim 5, wherein the second stent is deployed elastically by release from constraint.

8. A stenting system comprising:
a main branch stent having a side opening for alignment with an ostium of a side branch, wherein one or more first fluoroscopic markers are positioned near a periphery of the side opening; and
a side branch stent having an end adapted to be positioned at the side opening of the main branch stent, wherein one or more second fluoroscopic markers are positioned near the end to align with the marker(s) on the main branch stent when the side branch stent is properly positioned relative to the main branch stent at a vascular bifurcation.

9. A stenting system as in claim 8, wherein the main branch stent includes a side structure adapted to open laterally from the main branch stent through the ostium onto the side branch, wherein at least one of the fluoroscopic markers is positioned on the side structure.

10. A stenting system as in claim 9, wherein the side structure opens at least partially in response to radial expansion of the main branch stent.

11. A stenting system as in claim 10, wherein the side structure comprises at least one wing that opens laterally.

12. A stenting system as in claim 11, wherein the side structure comprises a plurality of wings which are interlinked to open together.

13. A stenting system as in claim 10, wherein the side structure includes a leverage mechanism that transfers displacement and expansion forces from the main body to open the side structure during radial expansion of the main body.

14. A stenting system as in claim 9, wherein the one or more fluoroscopic markers on the side branch stent are positioned to align with the at least one marker on the side structure after the side structure is opened.

15. A stenting system as in claim 8, further comprising a first catheter carrying the main branch stent and a second catheter carrying the side branch stent, wherein at least some of the second fluoroscopic markers are on the second catheter.
